(19) United States
(12) Patent Application Publication
(10) Pub. No.: US 2025/0246010 A1
(43) Pub. Date: Jul. 31, 2025

(54) CROSS-MODALITY PIXEL ALIGNMENT AND CELL-TO-CELL REGISTRATION ACROSS VARIOUS IMAGING MODALITIES

(71) Applicant: Genentech, Inc., South San Francisco, CA (US)

(72) Inventors: Rajiv Edillon JESUDASON, Oakland, CA (US); Tatiana NOVITSKAYA, Palo Alto, CA (US); Andries ZIJLSTRA, Alameda, CA (US); Mariya BARCH, San Francisco, CA (US)

(21) Appl. No.: 19/076,588

(22) Filed: Mar. 11, 2025

Related U.S. Application Data
(63) Continuation of application No. PCT/US2023/074176, filed on Sep. 14, 2023.
(60) Provisional application No. 63/407,077, filed on Sep. 15, 2022.

(51) Int. Cl. G06V 20/69; G01N 1/30; G01N 21/64; G01N 33/53; G16H 50/20

(52) U.S. Cl. CPC G06V 20/698; G01N 1/30; G01N 21/6486; G01N 33/53; G16H 50/20; G01N 2001/302

(57) ABSTRACT

A method implemented by one or more computer devices includes receiving a plurality of images of a set of tissue cells, the plurality of images comprising a first image including a first visualization modality and a second image including a second visualization modality. The method includes identifying a first tissue cell of the set of tissue cells in the first image and the first tissue cell in the second image, and performing a cell-to-cell registration process based on the first tissue cell identified in the first image and the first tissue cell identified in the second image. The cell-to-cell registration process includes matching of the first tissue cell identified in the first image to the first tissue cell identified in the second image. The method includes classifying the first tissue cell into a phenotype based on the cell-to-cell registration process, the phenotype partially indicative of a disease pathology.

### CROSS-REFERENCE TO RELATED APPLICATIONS

This application is a continuation of International Application No. PCT/US2023/074176, filed on Sep. 14, 2023, which claims priority to U.S. Provisional Patent Application No. 63/407,077, filed Sep. 15, 2022, entitled “Cross-Modality Pixel Alignment and Cell-to-Cell Registration Across Various Imaging Modalities,” the contents of which are hereby incorporated by reference in their entirety.

### TECHNICAL FIELD

This application relates generally to molecular annotation, and, more particularly, to resolving disparities in molecular annotation utilizing cross-modality pixel alignment and cell-to-cell registration across various imaging modalities.

### BACKGROUND

Digital pathology typically involves visualizing and analyzing digitized slides to ascertain whether variations occurring in tissue cells are due to disease, toxicity, and/or natural processes. The visualizations of tissue cells may generally include modalities consisting of dye-based visualization modalities, molecular-based visualization modalities, or probe-based visualization modalities. For example, dye-based visualizations, such as hematoxylin and eosin (H&E), may utilize the chemical properties of the dye molecules to bind to specific tissue cells, such as mucin, fats, and proteins. Similarly, molecular-based visualizations, such as immunohistochemistry (IHC), may utilize antibodies to bind to protein epitope targets with a high degree of specificity, and may be visualized, for example, utilizing 3,3′-diaminobenzidine or other similar organic compound to produce a brownish stain. IHC may also include other visualization colors, which may be combined, for example, to visualize multiple and different biomarkers.

Other molecular-based visualizations may include, for example, immunofluorescence (IF), which may utilize fluorescent dyes to multiplex and highlight a large number of different target cells (e.g., antibodies). Likewise, other molecular-based visualizations may include probe-based techniques that may be utilized to visualize, for example, messenger RNA (mRNA), micro RNA (miRNA), and DNA in one or more tissue cells utilizing bright-field or IF visualization modalities. Still, more recent visualization modalities may include, for example, hyperspectral imaging, which may be utilized to distinguish between dozens of different biomarkers labeled with different heavy metals and visualized utilizing imaging mass cytometry (IMC) or mass spectrometry (MS).

Thus, as may be appreciated from the foregoing, often different visualization modalities may be deployed to visualize and analyze the same target tissue cells based on the information pathologists, scientists, or clinicians are attempting to ascertain. For example, H&E stains may be well-suited for broadly visualizing specific tissue cells, such as cancer cells and proteins, while multiplex IHC (mxIHC) or multiplex IF (mxIF) may be well-suited to visualize and identify specific proteins and tissue cells. Various tissue cells (or assorted biological features) appear differently in different visualization modalities. For example, often a tissue cell image captured using one visualization modality may appear markedly different from an image of the same tissue cell captured using another visualization modality, and thus it may be challenging, or even counterintuitive, for either humans or computational-based models to recognize that the images portray the same tissue cell.

For example, while some visualization modalities may include spatial features for pathologists, scientists, and/or clinicians to easily identify or annotate target tissue cells, other visualization modalities may require computational-based modeling to identify and classify target tissue cells. Yet, without having verifiable annotations of target tissue cells for comparison and validation, even computational-based modeling may not accurately identify and classify target tissue cells. Still, even with conventional image registration techniques that may be suitable for aligning certain two-dimensional (2D) digital images, such conventional image registration techniques typically perform poorly when utilized to align individual tissue cells and/or other tissue cell features. Moreover, these conventional image registration techniques may typically require that the 2D images being aligned include the same imaging modality, and is thus technically unsuitable for aligning and keeping track of individual tissue cells across various imaging or visualization modalities. Accordingly, it may be useful to provide improved techniques to keep track of target tissue cells between different visualization modalities.

### SUMMARY

Embodiments of the present disclosure are directed toward one or more computing devices, methods, and non-transitory computer-readable media that may perform a cross-modality cell-to-cell registration process to identify. phenotype, and keep track of tissue cells captured utilizing various visualization modalities. The identification and phenotyping of tissue cells may use one visualization modality. The one visualization modality may be utilized as verifiable ground truth data for training one or more machine-learning models to classify and phenotype tissue cells captured utilizing another visualization modality. That is, in accordance with the presently disclosed embodiments, ground truth data for training one or more machine-learning models to classify and phenotype tissue cells for a particular visualization modality (e.g., in which tissue cells cannot be readily ascertained by observation) may be produced by relying on, for example, spatial features that may be readily ascertainable by observation of human experts (e.g., pathologists, scientists, clinicians, or other medical and scientific experts) with respect to a different visualization modality.

Indeed, by utilizing a cross-modality cell-to-cell registration process, one or more tissue cells or populations of tissue cells may be identified, classified, and phenotyped across various visualization modalities. In this way, pathologists, scientists, clinicians (e.g., oncologists), or other medical and scientific experts may more readily classify and phenotype immune cells (e.g., cancer cells, macrophages, regulatory T-cells (Tregs), CD8 cells, B lymphocytes, natural killer (NK) cells, fibroblasts, and so forth). This may engender, for example, earlier detection and diagnosis of non-Hodgkin's lymphoma (NHL) in patients, including both early and more progressive stages of NHL, such as follicular lymphoma (FL) and diffuse large B-cell lymphoma (DLBCL). The present cross-modality cell-to-cell registration techniques may further improve and advance the efficacy of targeted immunotherapies (e.g., monoclonal antibodies (mAbs). T-cell engaging bispecific antibodies (bsAbs), antibody-drug conjugates (ADCs), and so forth) for patients with NHL, including FL and DLBCL.

In certain embodiments, one or more computing devices may receive a number of images of a set of tissue cells, in which the number of images may include at least a first image including a first visualization modality and a second image including a second visualization modality. For example, in certain embodiments, the first visualization modality and the second visualization modality may be independently acquired by a whole slide imaging modality, microscopy modality, non-optical imaging modality. or spatial transcriptomics (ST) imaging modality. The whole slide imaging modality may be selected from bright-field or fluorescence imaging. The microscopy modality may be selected from bright-field microscopy, fluorescence microscopy, confocal microscopy, or high-content screening (HCS) microscopy, or synthetic image generation. The non-optical imaging modality may be selected from imaging mass cytometry (IMC) or myocardial perfusion imaging (MIBI). In some embodiments, the first visualization modality or the second visualization modality comprises a dye-based visualization modality. In some embodiments, the dye-based visualization modality may be selected from histological staining. fluorescence in situ hybridization (FISH), or immunofluorescence staining. In some embodiments, the dye-based visualization modality comprises hematoxylin and eosin (H&E) staining. In some embodiments, the first visualization modality or the second visualization modality may include immunostaining.

In certain embodiments, the one or more computing devices may identify regions of pixels in the first image and the second image, in which each of the regions of pixels corresponds to a respective tissue cell of the set of tissue cells. For example, in some embodiments. prior to identifying the regions of pixels in the first image and the second image, the one or more computing devices may align the number of images to vertically stack at least the first image and the second image. For example, in some embodiments, identifying the regions of pixels in the first image and the second image may include performing a nuclear segmentation of the regions of pixels in the first image and the second image to segment respective tissue cells of the set of tissue cells. In certain embodiments, the one or more computing devices perform a cell-to-cell registration process based on the identified regions of pixels, the cell-to-cell registration process including matching a first region of pixels corresponding to a first tissue cell in the first image to a second region of pixels corresponding to the first tissue cell in the second image.

For example, in certain embodiments, performing the cell-to-cell registration process may include performing a scale-invariant Fourier transform (SIFT) alignment of the first image and the second image, performing a tile-level alignment of the first image and the second image, the tile-level alignment comprising a matrix transformation of the SIFT alignment of the first image and the second image, and performing a tile-level segmentation of the tile-level aligned first image and second image. In some embodiments, the tile-level segmentation is performed to segment each tissue cell of the set of tissue cells in the first image and the second image. In certain embodiments, performing the cell-to-cell registration process further may further include performing an object-level cell registration based on the tile-level segmented tissue cells. In some embodiments, the object-level cell registration may be performed to match the first tissue cell in the first image to the first tissue cell in the second image. In certain embodiments, prior to performing the cell-to-cell registration process, the one or more computing devices may extract one or more features from the first image and the second image based on the identified regions of pixels. In some embodiments, one or more features are utilized to identify the first tissue cell in the first image and the first tissue cell in the second image.

In certain embodiments, the one or more computing devices may then classify the first tissue cell into a phenotype based on the cell-to-cell registration process. In some embodiments, the phenotype may be at least partially indicative of a disease pathology. For example, in some embodiments, classifying the first tissue cell into the phenotype may include classifying, based on one or more spatial features, the first tissue cell as a cancer cell, a plasma cell, a lymphocyte, a macrophage, or a fibroblast. In some embodiments, classifying the first tissue cell into the phenotype may include classifying, based on one or more molecular annotations, the first tissue cell as a cancer cell, a macrophage, a regulatory T-cell (Treg), a CD8 cell, a B lymphocyte, a natural killer (NK) cell, or a fibroblast. In some embodiments, the disease pathology may include a non-Hodgkin's lymphoma disease pathology, which may include follicular lymphoma (FL) or a diffuse large B-cell lymphoma (DLBCL). In certain embodiments, the one or more computing devices may then generate a phenotyping table based on the phenotype classification of the first tissue cell.

In certain embodiments, during a training phase, the one or more computing devices may determine a phenotype class label for the first tissue cell based on one or more spatial features associated with the first region of pixels corresponding to the first tissue cell, determine a correspondence between the first region of pixels corresponding to the first tissue cell and the second region of pixels corresponding to the first tissue cell based on the cell-to-cell registration process. Embodiments of the disclosure include training a model based on 1) the second region of pixels corresponding to the first tissue cell, and 2) the determined phenotype class label for the first tissue cell. In certain embodiments, during an inference phase, the one or more computing devices may input a third image of the plurality of images to the trained model. In some embodiments, the third image includes the second visualization modality. In certain embodiments, the one or more computing devices may utilize the trained model to identify a region of pixels in the third image corresponding to the first tissue cell, and output a predicted phenotype class label for the first tissue cell in the third image. In some embodiments, the predicted phenotype class label for the first tissue cell may correspond to the determined phenotype class label for the first tissue cell.

In some embodiments, the model may include one or more deep neural networks (DNNs). In certain embodiments, the one or more computing devices may determine ground truth data for training the model by mapping the phenotype classification of the first tissue cell to the first tissue cell in the first image and to the first tissue cell in the second image. In some embodiments, at least a subset of the ground truth data may include molecular annotated ground truth data. In another embodiment, at least a subset of the ground truth data may include human annotated ground truth data. In certain embodiments, the one or more computing devices may map the phenotype classification of the first tissue cell to the first tissue cell in the first image and to the first tissue cell in the second image using a phenotyping table.

### BRIEF DESCRIPTION OF THE DRAWINGS

One or more drawings included herein are in color in accordance with 37 CFR § 1.84. The color drawings are necessary to illustrate the invention. More specifically, FIGS. 1B, 1C, 3B, 3D, and 4A-7 are one or more high-resolution tissue cells captured utilizing various visualization modalities, all of which color plays a predominant role in enabling one skilled in the art to understand the invention and such color drawings are the only practical medium for disclosing the subject matter to be patented.

FIG. 1A illustrates an exemplary network of interacting computer systems, including a cell-to-cell registration system.

FIG. 1B illustrates a cell-to-cell registration system workflow for performing a cross-modality cell-to-cell registration process.

FIG. 1C illustrates a running example of a cross-modality cell-to-cell registration process to identify, phenotype, and keep track of tissue cells captured utilizing various visualization modalities.

FIG. 2A illustrates a flow diagram of a method for providing a cross-modality cell-to-cell registration process to identify, phenotype, and keep track of tissue cells captured utilizing various visualization modalities.

FIG. 2B illustrates another flow diagram of a method for providing a cross-modality cell-to-cell registration process to identify, phenotype, and keep track of tissue cells captured utilizing various visualization modalities.

FIG. 3A illustrates a flow diagram of a method for training one or more machine-learning models to classify and phenotype tissue cells in one visualization modality image utilizing class labels from a different visualization modality image as ground truth.

FIG. 3B illustrates a running example of training one or more machine-learning models to classify and phenotype tissue cells in one visualization modality image utilizing class labels from a different visualization modality image as ground truth.

FIG. 3C illustrates a flow diagram of a method for utilizing one or more machine-learning models trained to classify and phenotype tissue cells.

FIG. 3D illustrates a running example of utilizing one or more machine-learning models trained to classify and phenotype tissue cells.

FIGS. 4A and 4B illustrate annotations of immune cells on a whole slide image for five or more classes of immune cells.

FIGS. 4C and 4D illustrate annotations of immune cells for seven or more classes of immune cells and phenotype table.

FIGS. 5A-5C illustrate one or more graphical or implementation examples of a tissue cell matching example.

FIG. 6 illustrate one or more graphical or implementation examples of a cross-modality cell-to-cell registration process.

FIG. 7 illustrate one or more graphical or implementation examples of a cross-modality cell-to-cell registration process.

FIG. 8 illustrates a diagram of an example artificial intelligence (AI) architecture included as part of the network of interacting computer systems.

### DESCRIPTION OF EXAMPLE EMBODIMENTS

Embodiments of the present disclosure are directed toward one or more computing devices, methods, and non-transitory computer-readable media that may perform a cross-modality cell-to-cell registration process to identify, phenotype, and keep track of tissue cells captured utilizing various visualization modalities. Such a registration process may allow improved visualizations of various cells. The disclosed process may further permit the identification and phenotyping of tissue cells using one visualization modality may be utilized as verifiable ground truth data for training one or more machine-learning models to classify and phenotype tissue cells captured utilizing another visualization modality. That is, in accordance with the presently disclosed embodiments, ground truth data for training one or more machine-learning models to classify and phenotype tissue cells for a particular visualization modality (e.g., in which tissue cells cannot be readily ascertained by observation) may be produced by relying on, for example, spatial features that may be readily ascertainable by observation of human experts (e.g., pathologists, scientists, clinicians, or other medical and scientific experts) with respect to a different visualization modality.

Indeed, by utilizing the cross-modality cell-to-cell registration process, one or more tissue cells or populations of tissue cells may be identified, classified, and phenotyped across various visualization modalities. In this way, pathologists, scientists, clinicians (e.g., oncologists), or other medical and scientific experts may more readily classify and phenotype immune cells (e.g., macrophages, regulatory T-cells (Tregs), CD8 cells, B lymphocytes, natural killer (NK) cells, and so forth). This may engender, for example, earlier detection and diagnosis of non-Hodgkin's lymphoma (NHL) in patients, including both early and more progressive stages of NHL, such as follicular lymphoma (FL) and diffuse large B-cell lymphoma (DLBCL). The present cross-modality cell-to-cell registration process may further improve and advance the efficacy of targeted immunotherapies (e.g., monoclonal antibodies (mAbs), T-cell engaging bispecific antibodies (bsAbs), antibody-drug conjugates (ADCs), and so forth) for patients with NHL, including FL and DLBCL.

As used herein, “phenotype”, “phenotyping”, or “phenotyped” may be broadly understood to refer to an identification of one or more tissue cells or immune cells, a cell state of one or more tissue cells or immune cells, a cell program of one or more tissue cells or immune cells, a spatial location of one or more tissue cells or immune cells, a physical appearance of one or more tissue cells or immune cells, a count of one or more tissue cells or immune cells, or other similar human or machine perceptible feature or characteristic of one or more tissue cells or immune cells that may be utilized to classify the one or more tissue cells or immune cells across various visualization modalities.

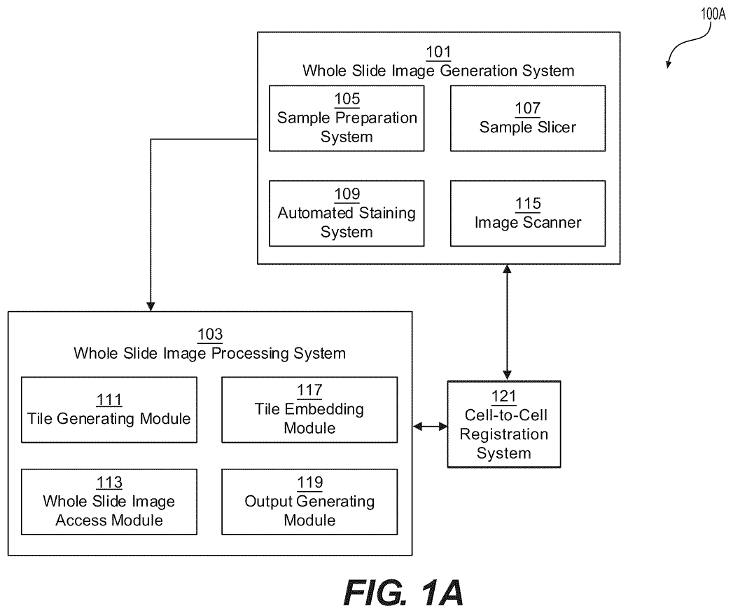

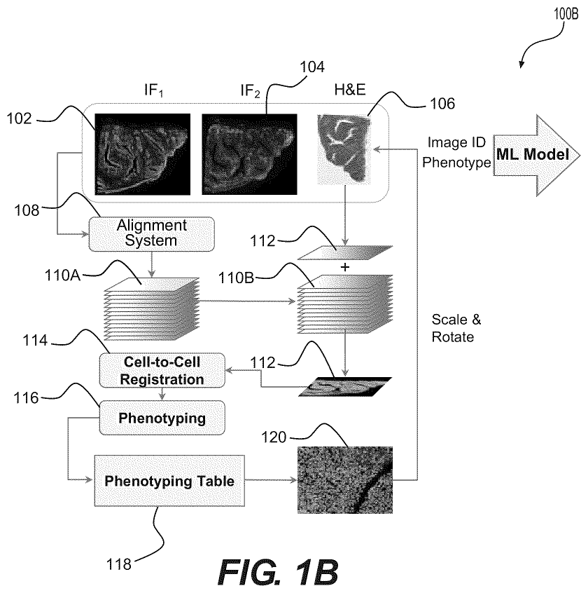

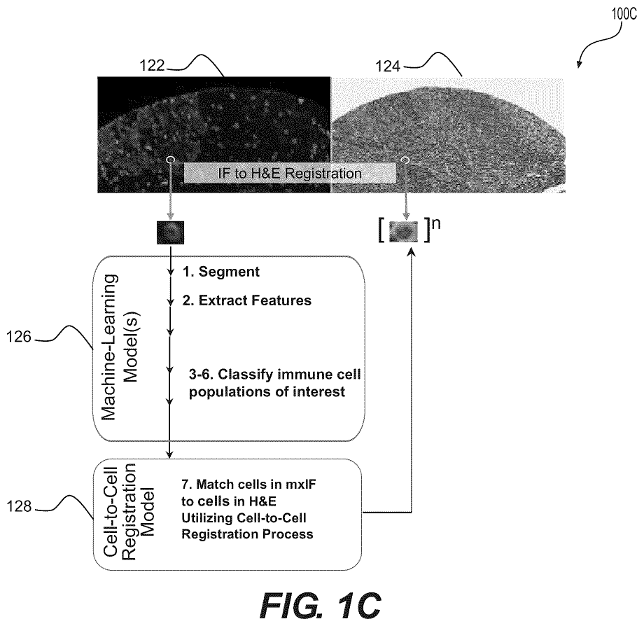

FIG. 1A illustrates a network 100A of interacting computer systems that may be suitable for performing a cross-modality cell-to-cell registration process to identify, phenotype, and keep track of tissue cells captured utilizing various visualization modalities, in accordance with the presently disclosed embodiments.

In certain embodiments, a whole slide image generation system 101 may generate one or more whole slide images or histopathology images, corresponding to a particular sample. For example, an image generated by whole slide image generation system 101 may include a stained section of a biopsy sample. As another example, an image generated by whole slide image generation system 101 may include a slide image (e.g., a blood film) of a liquid sample. As another example, an image generated by whole slide image generation system 101 may include fluorescence microscopy such as a slide image depicting fluorescence in situ hybridization (FISH) after a fluorescent probe has been bound to a target DNA or RNA sequence.

Some types of samples (e.g., samples including tissue) may be processed by a sample preparation system 105 to fix and/or embed the sample. Sample preparation system 105 may facilitate infiltrating the sample with a fixating agent (e.g., liquid fixing agent, such as a formaldehyde solution) and/or embedding substance (e.g., a histological wax). For example, a sample fixation sub-system may fix a sample by exposing the sample to a fixating agent for at least a threshold amount of time (e.g., at least 3 hours, at least 6 hours, or at least 13 hours). A dehydration sub-system may dehydrate the sample (e.g., by exposing the fixed sample and/or a portion of the fixed sample to one or more ethanol solutions) and potentially clear the dehydrated sample using a clearing intermediate agent (e.g., that includes ethanol and a histological wax). A sample embedding sub-system may infiltrate the sample (e.g., one or more times for corresponding predefined time periods) with a heated (e.g., and thus liquid) histological wax. The histological wax may include a paraffin wax and potentially one or more resins (e.g., styrene or polyethylene). The sample and wax may then be cooled, and the wax- infiltrated sample may then be blocked out.

In certain embodiments, a sample slicer 107 may receive the fixed and embedded sample and may produce a set of sections. Sample slicer 107 may expose the fixed and embedded sample to cool or cold temperatures. Sample slicer 107 may then cut the chilled sample (or a trimmed version thereof) to produce a set of sections. Each section may have a thickness that is (for example) less than 100 μm, less than 50 μm, less than 10 μm or less than 5 μm. Each section may have a thickness that is (for example) greater than 0.1 μm, greater than 1 μm, greater than 2 μm or greater than 4 μm.

In certain embodiments, an automated staining system 109 may facilitate staining one or more of the sample sections by exposing each section to one or more staining agents. Each section may be exposed to a predefined volume of staining agent for a predefined period of time. In some instances, a single section is concurrently or sequentially exposed to multiple staining agents. In certain embodiments, each of one or more stained sections may be presented to an image scanner 115, which may capture a digital image of the section. Image scanner 115 may include a microscope camera. The image scanner 115 may capture the digital image at multiple levels of magnification (e.g., using a 10× objective, 20× objective, 40× objective, and so forth). Manipulation of the image may be used to capture a selected portion of the sample at the desired range of magnifications.

It will be appreciated that one or more components of whole slide image generation system 101 may, in some instances, operate in connection with human operators. For example, human operators may move the sample across various sub-systems (e.g., of sample preparation system 105 or of whole slide image generation system 101) and/or initiate or terminate operation of one or more sub-systems, systems, or components of whole slide image generation system 101. In certain embodiments, whole slide image generation system 101 may transmit an image produced by image scanner 115 to a cell-to-cell registration system 121 in accordance with the presently-disclosed techniques. Although not illustrated, other intermediary devices (e.g., data stores of a server connected to the whole slide image generation system 101 or whole slide image processing system 103) may also be used.

The network 100A may be used in a variety of contexts where scanning and evaluation of histopathology images. such as whole slide images, are an essential component of the work. For example, whole slide image processing system 103 may process histopathology images, including whole slide images, to classify the digital pathology images and generate annotations for the digital pathology images and related output. A tile generating module 111 may define a set of tiles or patches for each digital pathology image. To define the set of tiles or patches, the tile generating module 111 may segment the digital pathology image into the set of tiles or patches. The tile generating module 111 may further define a tile or patch size depending on the type of abnormality being detected. For example, the tile generating module 111 may be configured with awareness of the type(s) of tissue abnormalities that the whole slide image processing system 103 will be searching for and may customize the tile or patch size according to the tissue abnormalities to optimize detection.

In certain embodiments, a tile embedding module 117 may generate an embedding for each tile or patch in a corresponding feature embedding space. The embedding may be represented by the whole slide image processing system 103 as a feature vector for the tile or patch. The tile embedding module 117 may utilize a neural network (e.g., one or more convolution neural network (CNNs)) to generate a feature vector that represents each tile or patch of the image. In particular embodiments, the tile embedding neural network may be based on a residual neural network (ResNet) image classification network trained on a data set based on natural (e.g., non-medical) images. such as the ImageNet dataset. In other embodiments, the tile embedding network utilized by the tile embedding module 117 may be an embedding network customized to handle large numbers of tiles or patches of large format images, such as digital pathology whole slide images.

In certain embodiments, a whole slide image access module 113 may manage requests to access whole slide images from other modules of the whole slide image processing system 103 and the cell-to-cell registration system 121. For example, in some embodiments, the whole slide image access module 113 may receive requests to access, for example, a multiplex immunofluorescence (mxIF) image, a hematoxylin and eosin (H&E) image, or other imaging or visualization modality to provide to the cell-to-cell registration system 121. For example, in one embodiment, an output generating module 119 of the whole slide image processing system 103 may generate an output corresponding to the accessed or requested mxIF image and H&E image. The output generating module 119 may then provide the output to the cell-to-cell registration system 121 for performing a cross-modality cell-to-cell registration process to identify, phenotype, and keep track of tissue cells captured by the mxIF image and the H&E image, in accordance with the presently disclosed embodiments.

FIG. 1B illustrates a cell-to-cell registration system workflow 100B for performing a cross-modality cell-to-cell registration process to identify, phenotype, and keep track of tissue cells captured utilizing various visualization modalities, in accordance with the presently disclosed embodiments. In certain embodiments, the system workflow 100B may begin with accessing a multiplex immunofluorescence (mxIF) image 102, an mxIF image 104, and a hematoxylin and eosin (H&E) image 106. For example, in certain embodiments, the mxIF image 102, the mxIF image 104, and the H&E image 106 may each include one or more tissue cells. Specifically, in accordance with the presently disclosed embodiments, the mxIF image 102, the mxIF image 104, and the H&E image 106 may each include the same exact tissue cells captured by different visualization modalities. Indeed, while the present cross-modality cell-to-cell registration techniques may be discussed herein primarily with respect to mxIF and H&E visualization modalities, it should be appreciated that the present cross-modality cell-to-cell registration techniques may be applied to any of various visualization modalities, such as immunohistochemistry (IHC), fluorescence in situ hybridization (FISH), bright-field microscopy, imaging mass cytometry (IMC), myocardial perfusion imaging (MIBI), among various other visualization modalities.

For example, in one embodiment, the mxIF image 102 (e.g., “IF₁) and the mxIF image 104 (e.g., “IF₂) may each include a molecular-based visualization, such as multiplex immunofluorescence (mxIF), which may utilize fluorescent dyes to multiplex and highlight a large number of different target cells, for example. For example, in some embodiments, the mxIF image 102 (e.g., “IF₁) and the mxIF image 104 (e.g., “IF₂) may each include an mxIF image of the tissue cells captured utilizing a cyclic immunofluorescence process, which may be utilized to generate highly multiplexed images using a cycling process (e.g., a cycle) in which IF images are repeatedly collected of the same tissue cells and ultimately assembled.

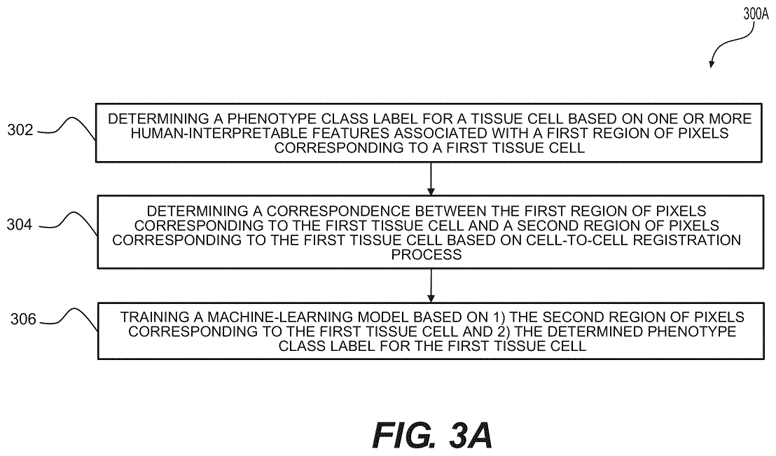

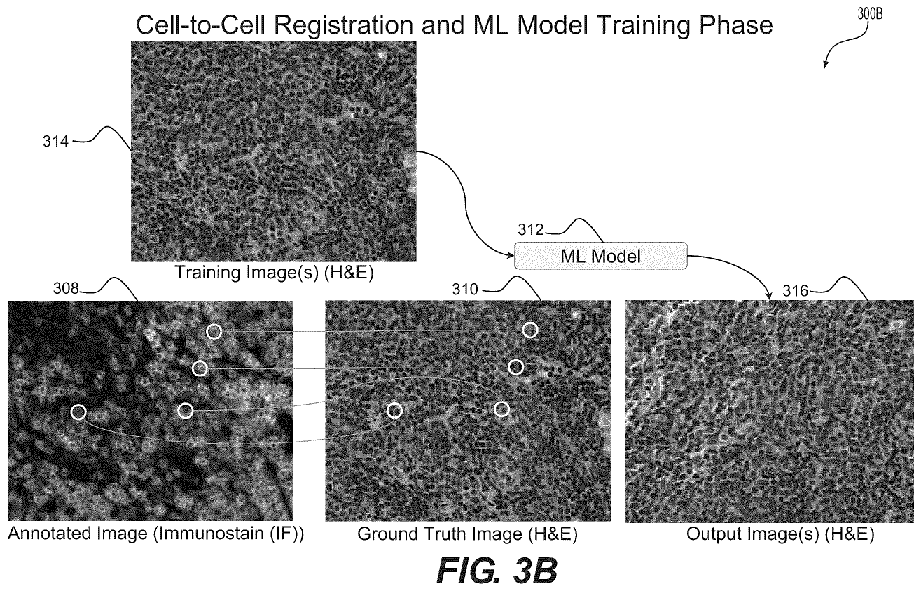

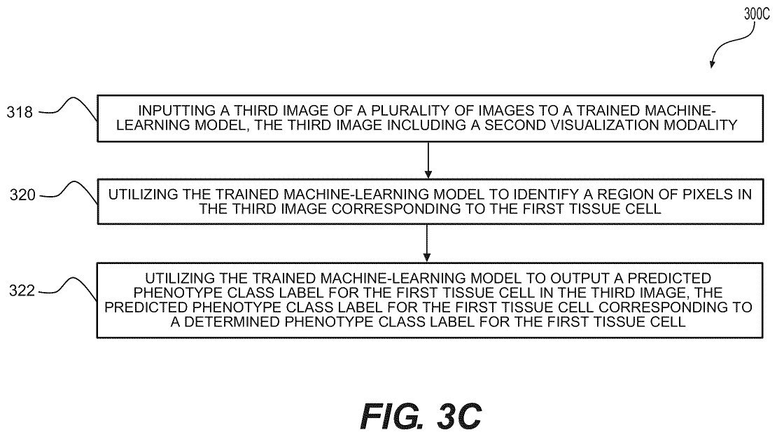

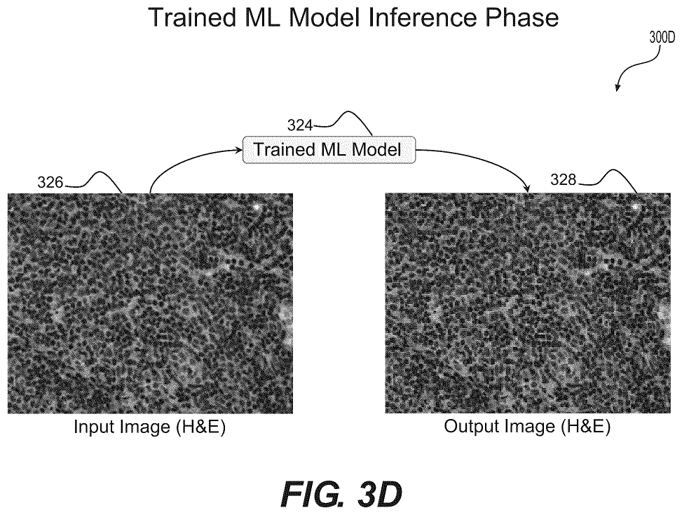

Thus, in one embodiment, the mxIF image 102 (e.g., “IF₁) may be representative of an mxIF image captured in a first cycle of the cycling process and the mxIF image 104 (e.g., “IF₂) may be representative of an mxIF image captured in a second cycle of the cycling process. Similarly, in one embodiment, the H&E image 106 may include a dye-based visualization, such as hematoxylin and eosin (H&E), which may utilize the chemical properties of the dye molecules to bind to one or more specific tissue cells. In certain embodiments, as will be further appreciated below with respect to FIG. 3A-3D, the mxIF image 102 (e.g., “IF₁) and the mxIF image 104 (e.g., “IF₂) may include one or more spatial features (e.g., any features suitable for informing or ascertaining the phenotype of a cell by its spatial organization with respect to neighboring cells or its position within a tissue or region of a tissue) suitable for allowing a pathologist, scientist, or clinician (e.g., oncologist) to manually label one or more tissue cells to be matched utilizing a cell-to-cell registration process to the corresponding one or more tissue cells in the H&E image 106, which may not include spatial features.

In certain embodiments, the mxIF image 102, the mxIF image 104, and the H&E image 106 may be then inputted into an alignment system 108. For example, in certain embodiments, the alignment system 108 may include, for example, any process that may be utilized to vertically align (e.g., pixel-to-pixel alignment or whole slide image alignment by way of cross-correlation) the mxIF image 102, the mxIF image 104, and the H&E image 106 into a vertical stack 110A. In some embodiments, the alignment system 108 may perform a scale-invariant Fourier transform (SIFT) alignment of the mxIF image 102, the mxIF image 104, and the H&E image 106. In another embodiment, the alignment system 108 may perform a tile-level alignment of the mxIF image 102, the mxIF image 104, and the H&E image 106. In certain embodiments, the tile-level alignment may be performed subsequent to, or in conjunction with. the SIFT alignment. For example, in one embodiment, the tile-level alignment may include a matrix transformation of the SIFT alignment of the mxIF image 102. the mxIF image 104, and the H&E image 106.

In certain embodiments, the system workflow 100B may continue with the H&E image 106 may be scaled and rotated. For example, in one embodiment, the scaled and rotated H&E image 106 may be placed atop an updated vertical stack 110B. In certain embodiments, the system workflow 100B may continue with an image segmentation 112 of the updated vertical stack 110B. For example, in some embodiments, the image segmentation 112 may include, for example, nuclear segmentation that may be utilized to segment pixels of the mxIF image 102, the mxIF image 104, and the H&E image 106 within the updated vertical stack 110B, for example, along boundaries of nuclei of individual tissue cells. In another embodiment, the image segmentation 112 may include, for example, semantic segmentation (e.g., pixel-wise image segmentation) that may be utilized to segment annotate pixels of the mxIF image 102, the mxIF image 104, and the H&E image 106 within the updated vertical stack 110B. for example. on pixel-by-pixel basis. In certain embodiments, the system workflow 100B may then proceed with performing a cross-modality cell-to-cell registration process 114 in accordance with the presently disclosed techniques.

In certain embodiments, the cross-modality cell-to-cell registration process 114 may include, for example, an object-level cell registration to match individual tissue cells across the mxIF image 102, the mxIF image 104, and the H&E image 106 within the segmented and updated vertical stack 110B. For example, in some embodiments, the object-level cell registration may include matching, for example, polygons (e.g., two-dimensional (2D) or three-dimensional (3D) polygons) multi-directionally based on an identification of overlapping tissue cells and/or intersecting tissue cells across the mxIF image 102, the mxIF image 104, and the H&E image 106 within the segmented and updated vertical stack 110B. In some embodiments, the polygons with multiple matches may be also tracked and recorded (e.g., into the phenotyping table 118).

In certain embodiments, based on the cross-modality cell-to-cell registration process 114. the system workflow 100B may then proceed with performing phenotyping process 116 of the one or more matched tissue cells. For example, in some embodiments, the phenotyping process 116 may include classifying the one or more matched tissue cells, for example, into one or more classes of immune cells, such as macrophages, regulatory T-cells (Tregs), CD8 cells, B lymphocytes, natural killer (NK) cells, and so forth. In certain embodiments, the system workflow 100B may then proceed with storing the aforementioned data into a phenotyping table 118. For example, in some embodiments, the phenotyping table 118 may include, for example, a record of the matched and identified tissue cells determined based on the cross-modality cell-to-cell registration process 114. In certain embodiments, the phenotyping table 118 may be then utilized to label an image 120 (e.g., H&E image), which may be then utilized in downstream tasks to train one or more machine-learning models to classify various immune cells (e.g., macrophages, Tregs, CD8 cells, a B lymphocytes, NK cells, and so forth) in accordance with the present embodiments. For example, in one embodiment, the phenotyping table 118 may include immune cell phenotype, the immune cell activation state, the image identification or label, the shape, and location of the tissue cells of interest.

FIG. 1C illustrates a running example 100C of a cross-modality cell-to-cell registration process to identify, phenotype, and keep track of tissue cells or immune cells captured utilizing various visualization modalities, in accordance with the presently disclosed embodiments. As depicted, in one embodiment, the running example 100C of a cross-modality cell-to-cell registration process may be described with respect to an mxIF image 122 and an H&E image 124. As illustrated, mxIF image 122 may be passed to one or more machine-learning models 126, which may be utilized, for example, to segment the mxIF image 122, extract one or more features of interest corresponding to one or more tissue cells, and classify and phenotype the one or more tissue cells. For example, in some embodiments, the one or more tissue cells may include one or more populations of immune cells (e.g., cancer cells, macrophages, Tregs, CD8 cells, a B lymphocytes, NK cells, fibroblasts, and so forth). In certain embodiments, the classified and phenotyped one or more tissue cells in the mxIF image 122 may be then matched to the corresponding one or more tissue cells in the H&E image 124 utilizing a cell-to-cell registration model 128.

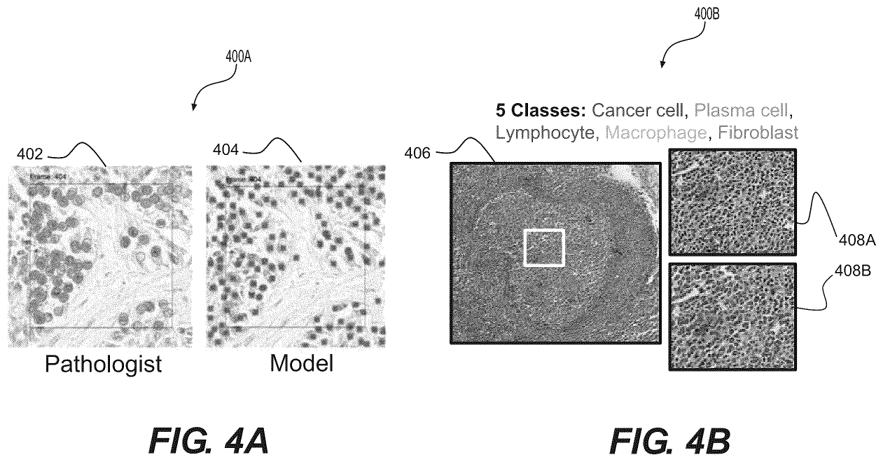

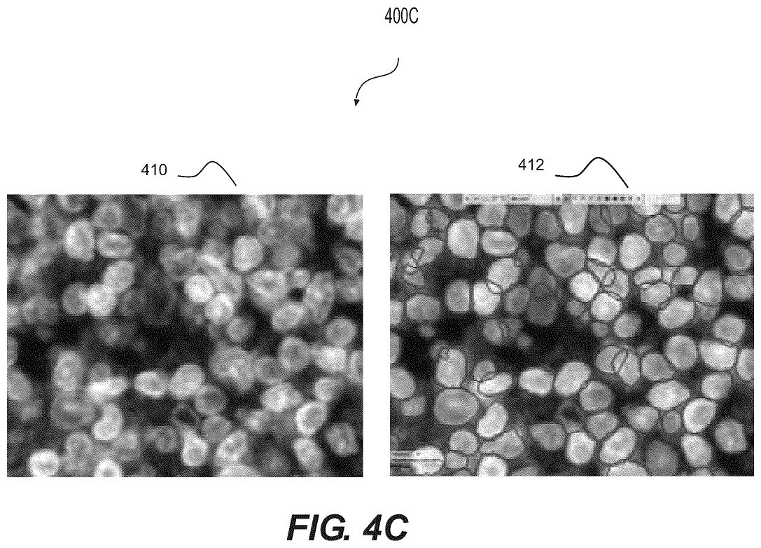

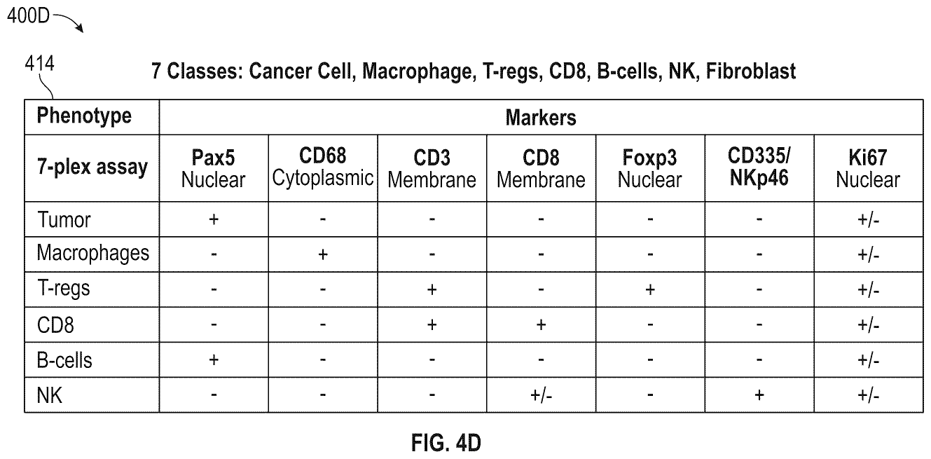

Specifically, in accordance with the presently disclosed embodiments, the cell-to-cell registration model 128 may perform a cell-to-cell registration process suitable for matching the one or more populations of immune cells identified in the mxIF image 122 to the corresponding immune cells in the H&E image 124. The H&E image 124 may be then utilized as ground truth data to train one or more machine-learning models to classify immune cells or other tissue cells. Specifically, as will be further appreciated with respect to FIGS. 3A-3D and 4A-4D, the one or more populations of immune cells identified in the mxIF image 122 may be classified and phenotyped based on one or more spatial features (e.g., any features suitable for informing or ascertaining the phenotype of a cell by its spatial organization with respect to neighboring cells or its position within a tissue or region of a tissue), for example, by a human annotator (e.g., scientist, pathologist, clinician, or other medical or scientific expert). Thus, by then matching the one or more populations of immune cells to the corresponding immune cells in the H&E image 124, the classification and phenotyping can be verifiably trusted (e.g., 90%-100% confidence score) as ground truth data for accurately training the one or more machine-learning models to classify immune cells or other tissue cells in H&E image 124.

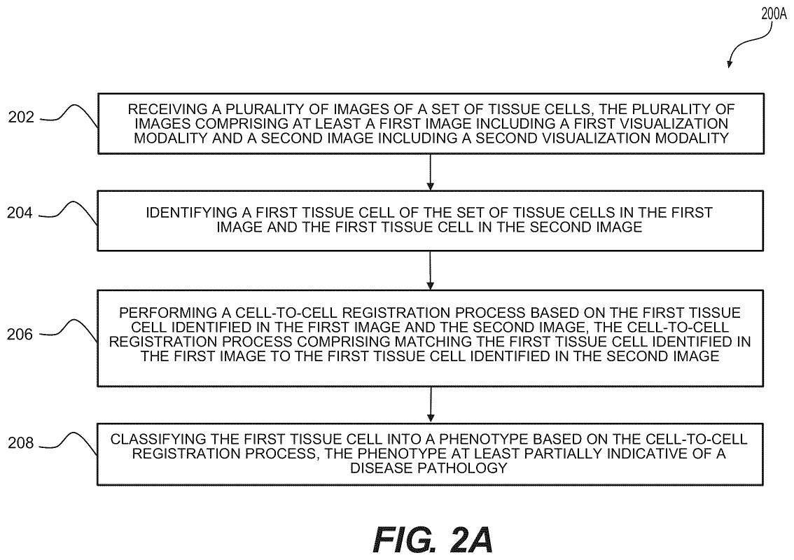

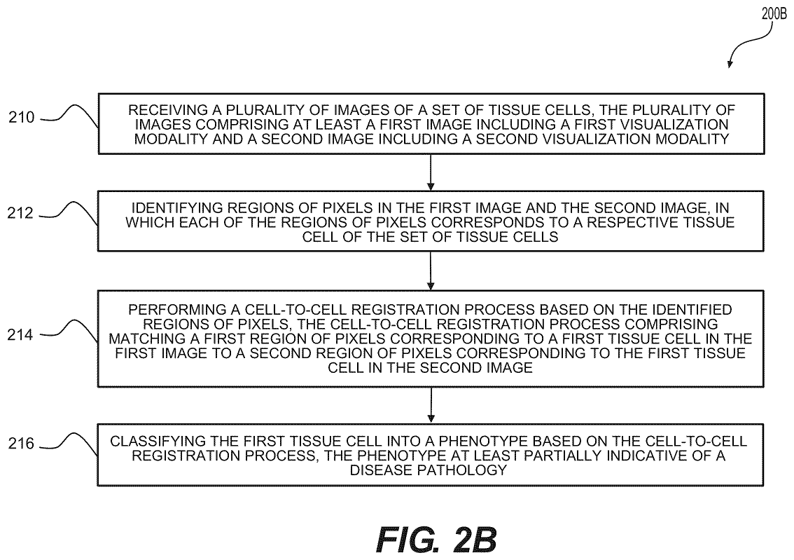

FIG. 2A illustrates a flow diagram of a method 200A for providing a cross-modality cell-to-cell registration process to identify, phenotype, and keep track of tissue cells captured utilizing various visualization modalities, in accordance with the presently disclosed embodiments. The method flow diagram 200A may be performed utilizing one or more processing devices network 100A that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), a neuromorphic processing unit (NPU), or any other processing device(s) that may be suitable for processing various omics data and making one or more decisions based thereon), software (e.g., instructions running/executing on one or more processors), firmware (e.g., microcode), or some combination thereof.

The method 200A may begin at block 202 with one or more processing devices receiving a plurality of images of a set of tissue cells. the plurality of images including at least a first image including a first visualization modality and a second image including a second visualization modality. For example, in one embodiment, the first image may include an mxIF visualization modality and the second image may include an H&E visualization modality. The method 200A may include block 204, with one or more processing devices identifying a first tissue cell of the set of tissue cells in the first image and the first tissue cell in the second image. The method 200A may also include block 206 in which one or more processing devices performing a cell-to-cell registration process based on the first tissue cell identified in first image and the first tissue cell identified in the second image. For example, in some embodiments, the cell-to-cell registration process may include matching the first tissue cell in the first image to the first tissue cell in the second image. The method 200A may further include block 208, with one or more processing devices classifying the first tissue cell into a phenotype based on the cell-to-cell registration process, the phenotype being at least partially indicative of a disease pathology. For example, in certain embodiments. the cell-to-cell registration process may include matching one or more populations of immune cells identified in the mxIF image to the corresponding immune cells in the H&E image.

As noted above, by utilizing the cross-modality cell-to-cell registration process, one or more tissue cells or populations of tissue cells may be identified, classified, and phenotyped across various visualization modalities. In this way, pathologists, scientists, clinicians (e.g., oncologists), or other medical and scientific experts may more readily classify and phenotype immune cells (e.g., macrophages, regulatory T-cells (Tregs), CD8 cells, B lymphocytes, natural killer (NK) cells, and so forth). This may engender, for example, earlier detection and diagnosis of non-Hodgkin's lymphoma (NHL) in patients, including both early and more progressive stages of NHL, such as follicular lymphoma (FL) and diffuse large B-cell lymphoma (DLBCL). The present cross-modality cell-to-cell registration techniques may further improve and advance the efficacy of targeted immunotherapies (e.g., monoclonal antibodies (mAbs), T-cell engaging bispecific antibodies (bsAbs), antibody-drug conjugates (ADCs), and so forth) for patients with NHL, including FL and DLBCL.

FIG. 2B illustrates another flow diagram of a method 200B for providing a cross-modality cell-to-cell registration process to identify, phenotype, and keep track of tissue cells captured utilizing various visualization modalities, in accordance with the presently disclosed embodiments. The method 200B may be performed utilizing one or more processing devices network 100A that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), a neuromorphic processing unit (NPU), or any other processing device(s) that may be suitable for processing various omics data and making one or more decisions based thereon), software (e.g., instructions running/executing on one or more processors), firmware (e.g., microcode), or some combination thereof.

The method 200B may begin at block 210 with one or more processing devices receiving a plurality of images of a set of tissue cells, the plurality of images including at least a first image including a first visualization modality and a second image including a second visualization modality. For example, in one embodiment, the first image may include an mxIF visualization modality and the second image may include an H&E visualization modality. The method 200B may then continue at block 212 with one or more processing devices identifying regions of pixels in the first image and the second image, wherein each of the regions of pixels corresponds to a respective tissue cell of the set of tissue cells. For example, in some embodiments, nuclear segmentation that may be utilized to segment pixels of the mxIF image and the H&E image, for example, along boundaries of nuclei of individual tissue cells. The method 200B may include block 214, with one or more processing devices performing a cell-to-cell registration process based on the identified regions of pixels. For example, in certain embodiments, the cell-to-cell registration process may include matching a first region of pixels corresponding to a first tissue cell in the first image to a second region of pixels corresponding to the first tissue cell in the second image. The method 200B may include block 216, with one or more processing devices classifying the first tissue cell into a phenotype based on the cell-to-cell registration process, the phenotype being at least partially indicative of a disease pathology. For example, in certain embodiments, the cell-to-cell registration process may include matching one or more populations of immune cells identified in the mxIF image to the corresponding immune cells in the H&E image.

As noted above, by utilizing the cross-modality cell-to-cell registration process, one or more tissue cells or populations of tissue cells may be identified, classified, and phenotyped across various visualization modalities. In this way, pathologists, scientists, clinicians (e.g., oncologists), or other medical and scientific experts may more readily classify and phenotype immune cells (e.g., macrophages, regulatory T-cells (Tregs), CD8 cells, B lymphocytes, natural killer (NK) cells, and so forth). This may engender, for example, earlier detection and diagnosis of non-Hodgkin's lymphoma (NHL) in patients. including both early and more progressive stages of NHL, such as follicular lymphoma (FL) and diffuse large B-cell lymphoma (DLBCL). The present cross-modality cell-to-cell registration techniques may further improve and advance the efficacy of targeted immunotherapies (e.g., monoclonal antibodies (mAbs). T-cell engaging bispecific antibodies (bsAbs). antibody-drug conjugates (ADCs), and so forth) for patients with NHL, including FL and DLBCL.

FIG. 3A illustrates a flow diagram of a method 300A for training one or more machine-learning models to classify and phenotype tissue cells (e.g., immune cells) in one visualization modality image utilizing class labels from a different visualization modality image as ground truth, in accordance with the presently disclosed embodiments. The flow diagram 300A may be performed utilizing one or more processing devices network 100A that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), neuromorphic processing unit (NPU), or any other processing device(s) that may be suitable for processing various omics data and making one or more decisions based thereon), software (e.g., instructions running/executing on one or more processors), firmware (e.g., microcode), or some combination thereof.

The method 300A may begin at block 302 with one or more processing devices determining a phenotype class label for a first tissue cell based on one or more spatial features associated with a first region of pixels corresponding to the first tissue cell. The method 300A may include at block 304 the one or more processing devices determining a correspondence between the first region of pixels corresponding to the first tissue cell and a second region of pixels corresponding to the first tissue cell based on a cell-to-cell registration process. The method 300A may include at block 306 the one or more processing devices training a machine-learning model based on 1) the second region of pixels corresponding to the first tissue cell, and 2) the determined phenotype class label for the first tissue cell.

FIG. 3B illustrates a running example 300B of training one or more machine-learning models to classify and phenotype tissue cells (e.g., immune cells) in one visualization modality image utilizing class labels from a different visualization modality image as ground truth, in accordance with the presently disclosed embodiments. FIG. 3B is a running example of the process illustrated and described above with respect to FIG. 3A, As depicted, in one embodiment, the running example 300B of a cross-modality cell-to-cell registration process may be described with respect to an mxIF image 308 and an H&E image 310. However, it should be appreciated that the present techniques may be applied between any of visualization modalities.

As illustrated. one or more tissue cells in the mxIF image 308 may be annotated based on one or more spatial features (e.g., any features suitable for informing or ascertaining the phenotype of a cell by its spatial organization with respect to neighboring cells or its position within a tissue or region of a tissue), and then matched to the corresponding one or more tissue cells in the H&E image 310 (e.g., as illustrated by the lines with open circles extending between the mxIF image 308 and the H&E image 310). For example, as generally depicted by FIG. 3B, a human annotator (e.g., scientist, pathologist, clinician, or other medical or scientific expert) may observe the mxIF image 308 and classify and label one or more tissue cells or populations of tissue cells (e.g., a cancer cell, a plasma cell, a lymphocyte, a macrophage, a fibroblast, and so forth) based on, for example, one or more spatial features (e.g., varying colors of pixels of tissue cells, tissue cell count and density, tissue cell proportion and proximity, tissue cell area and multiplicity, and/or other features suitable for informing the phenotype of a cell by its spatial organization with respect to neighboring cells or its position within a tissue or region of a tissue).

In certain embodiments, once the one or more tissue cells or populations of tissue cells in the mxIF image 308 are classified and labeled (e.g., a cancer cell, a plasma cell, a lymphocyte, a macrophage, a fibroblast, and so forth), the corresponding one or more tissue cells or populations of tissue cells (e.g., the same tissue cells captured utilizing a different visualization modality) within the H&E image 310 may be then matched thereto in accordance with a cross-modality cell-to-cell registration process as described herein. Specifically, the corresponding one or more tissue cells or populations of tissue cells within the H&E image 310 are labeled (e.g., “this tissue cell within the H&E image 310 is a lymphocyte”; “that tissue cell within the H&E image 310 is a fibroblast”; “this other tissue cell within the H&E image 310 is a macrophage”; and so forth) based on information known and determined from the mxIF image 308.

In this way, even though the H&E image 310 may not itself include spatial features for easily determining and classifying tissue cells, by matching the tissue cells or populations of tissue cells to those within the mxIF image 308, the H&E image 310 may be rendered as proficient and accurate ground truth data for training one or more machine-learning models 312 to classify and phenotype tissue cells or immune cells (e.g., cancer cells, macrophages, Tregs, CD8 cells, a B lymphocytes, NK cells, fibroblasts, and so forth). That is, in some embodiments, the present cross-modality cell-to-cell registration process may allow the H&E image 310 to be molecularly annotated, so as to be rendered verifiably trusted ground truth data (e.g., as opposed to relying on spatial features observed by human annotators, particularly when certain spatial features may be present in one visualization modality and not present in other visualization modalities).

In certain embodiments, the one or more machine-learning models 312 (e.g., a deep neural network (DNN), a convolutional neural network (CNN), a fully-connected neural network (FCNN), and so forth) may be then trained (e.g., by way of supervised machine-learning). For example, during the training phase, the one or more machine-learning models 312 may be provided a data set of training image(s) 314. The training image(s) 314 may be an image having a first visualization modality or a second visualization modality (e.g., thousands of H&E training images). For example, the training image(s) 314 may be H&E training image(s). In some embodiments, the data set of H&E training image(s) 314 may be annotated for training the one or more machine-learning models 312 to identify and classify tissue cells based on the class labels determined and known from the mxIF image 308 (a different visualization modality). In certain embodiments, the one or more machine-learning models 312 may generate one or more predictions of class labels for the output image(s) 316. In certain embodiments, the one or more predictions of class labels for the output image(s) 316 may be then compared to the H&E image 310 (e.g., ground truth) and utilized to iteratively update (e.g., by way of backpropagation or a loss calculated between the one or more predictions of class labels for the output image(s) 316 and the ground truth H&E image 310) the one or more machine-learning models 312 until sufficiently trained (e.g., predicting class labels for the tissue cells with an accuracy of 0.8, 0.9, or higher on a scale of 0.0 to 1.0) to classify and phenotype tissue cells in H&E images.

FIG. 3C illustrates a flow diagram of a method 300C for utilizing one or more machine-learning models trained to classify and phenotype tissue cells (e.g., immune cells), in accordance with the presently disclosed embodiments. The method 300C may be performed utilizing one or more processing devices network 100A that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), a neuromorphic processing unit (NPU), or any other processing device(s) that may be suitable for processing various omics data and making one or more decisions based thereon), software (e.g., instructions running/executing on one or more processors), firmware (e.g., microcode), or some combination thereof.

The method 300C may begin at block 318 with one or more processing devices inputting a third image of a plurality of images to a trained machine-learning model, the third image including the second visualization modality. The flow diagram 300C may then include at block 320 the one or more processing devices utilizing the trained machine-learning model to identify a region of pixels in the third image corresponding to the first tissue cell. The flow diagram 300C may include at block 322 the one or more processing devices utilizing the trained machine-learning model to output a predicted phenotype class label for the first tissue cell in the third image, the predicted phenotype class label for the first tissue cell corresponding to the determined phenotype class label for the first tissue cell.

FIG. 3D illustrates a running example 300D of utilizing one or more machine-learning models trained to classify and phenotype tissue cells. in accordance with the presently disclosed embodiments. FIG. 3C is a running example of the process illustrated and described above with respect to FIG. 3D. As depicted, in certain embodiments, during the inference phase, the one or more trained machine-learning models 324 (e.g., trained as discussed above with respect to FIGS. 3A and 3B) may receive an input H&E image 326. In certain embodiments, based on the input H&E image 326, the one or more trained machine-learning models 324 may then generate an output H&E image 328 including one or more predicted class labels (e.g., cancer cells, macrophages, Tregs, CD8 cells, B lymphocytes, NK cells, fibroblasts, and so forth) for one or more tissue cells or populations of tissue cells within the output H&E image 328.

FIGS. 4A and 4B illustrate annotations of tissue cells or immune cells on a whole slide image for five or more classes of tissue cells or immune cells (e.g., cancer cells, plasma cells. lymphocytes, macrophages, fibroblasts, and so forth), in accordance with the presently disclosed embodiments. For example, as depicted, FIG. 4A illustrates annotations of immune cells as performed by a human annotator-based classification and labelling 402 as compared to model-based classification and labelling 404. FIG. 4B illustrates an H&E image 406, illustrating classifications of more classes of tissue cells or immune cells (e.g., cancer cells, plasma cells, lymphocytes, macrophages, fibroblasts, and so forth) labeled by color. For example, in certain embodiments, the magnified portion 408A of the H&E image 406 may illustrate one or more tissue cells or population of one or more tissue cells as unlabeled. while the magnified portion 408B of the H&E image 406 may illustrate the one or more tissue cells or population of one or more tissue cells as labeled (e.g., as illustrated by the individually colored tissue cells corresponding to cancer cells, plasma cells, lymphocytes, macrophages, and fibroblasts, respectively) in accordance with the presently disclosed techniques.

FIGS. 4C and 4D illustrate annotations of tissue cells or immune cells for seven or more classes of tissue cells or immune cells (e.g., cancer cells, macrophages, Tregs, CD8 cells, B lymphocytes, NK cells, fibroblasts, and so forth) and phenotype table, in accordance with the presently disclosed embodiments. For example, as depicted. FIG. 4C illustrates annotations of immune cells as performed by a human annotator-based classification and labelling 410 as compared to model-based classification and labelling 412. FIG. 4D illustrates a table 414 illustrating classifications of more phenotype classes of tissue cells or immune cells (e.g., cancer cells, plasma cells, lymphocytes, macrophages, fibroblasts, and so forth) labeled by color.

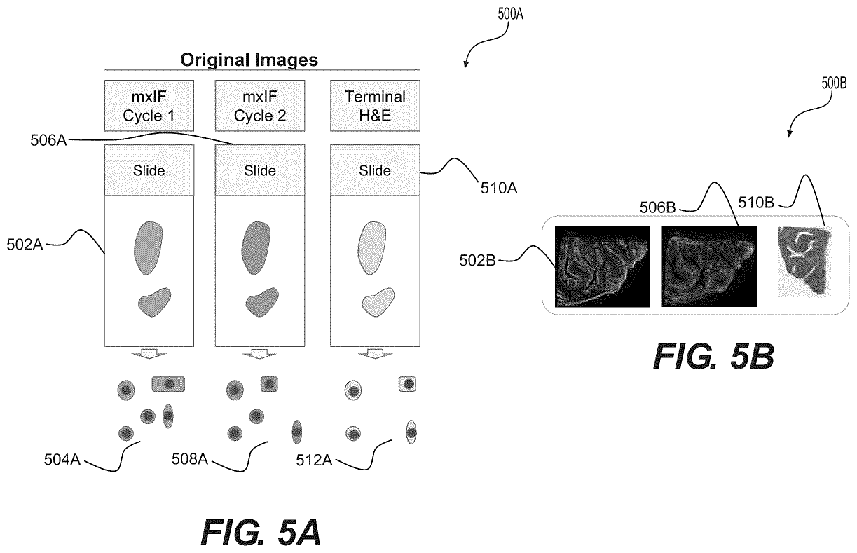

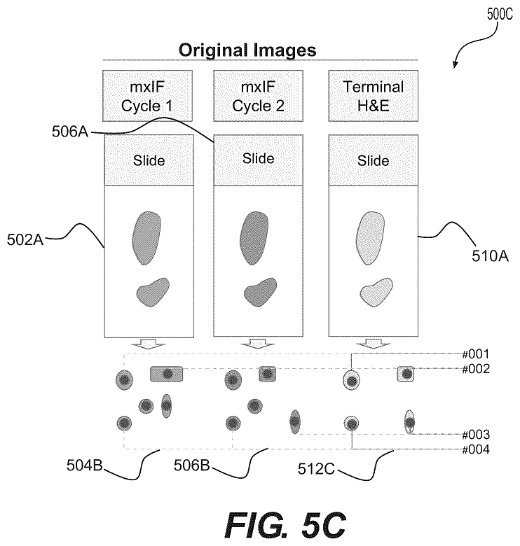

FIGS. 5A-5C illustrate one or more graphical or implementation examples of a tissue cell matching example, in accordance with the presently disclosed embodiments. For example, original images 500A of an mxIF slide 502A, an mxIF slide 506A, and an H&E slide 510A may include one or more tissue cells. It should be appreciated that the tissue cells 504A, the tissue cells 508A, and the tissue cells 512A may be the same exact tissue cells captured by different visualization modalities. For example, as depicted by the mxIF slide 502A, the one or more tissue cells 504A may be at different orientations, alignments, proximities, and so forth on one or more of the mxIF slide 502A, the mxIF slide 506A, and the H&E slide 510A.

FIG. 5B illustrates real-world images 500B of an mxIF image 502B corresponding to the mxIF slide 502A, an mxIF image 506B corresponding to the mxIF slide 506A, and an H&E image 510B corresponding to the H&E slide 510A. FIG. 5C illustrates a graphical example of the cell-to-cell registration process as described herein. Specifically, as depicted by images 500C of FIG. 5C, each of the tissue cells 504C, 508C, and 512C (e.g., tissue cell “001,” tissue cell “002”, tissue cell “003”, and tissue cell “004”) may be matched and tracked between the mxIF slide 502A, the mxIF slide 506A, and the H&E slide 510A, such that a phenotyping or classification of tissue cells on one or more of the mxIF slide 502A, the mxIF slide 506A, and the H&E slide 510A may be assumed as representative the other ones of the mxIF slide 502A, the mxIF slide 506A, and the H&E slide 510A.

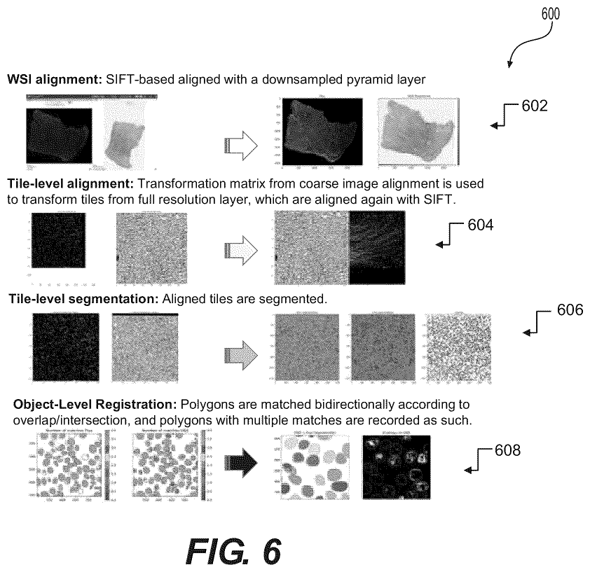

FIG. 6 illustrate one or more graphical or implementation examples 600 of a cross-modality cell-to-cell registration process, in accordance with the presently disclosed embodiments. Specifically, the cross-modality cell-to-cell registration process as illustrated by the one or more graphical or implementation examples 600 correspond to the cell-to-cell registration process 114 as discussed above with respect to FIG. 1A. For example, the cross-modality cell-to-cell registration process includes performing (602) a scale-invariant Fourier transform (SIFT) alignment of cross-modality visualization images (e.g., SIFT-based aligned with a down-sampled pyramid layer), and performing (604) a tile-level alignment of the cross-modality visualization images, including a matrix transformation of the SIFT alignment of the cross-modality visualization images (e.g., a matrix transformation from coarse image alignment is used to transform tiles from a full resolution layer, which is aligned again with SIFT).

In certain embodiments, the cross-modality cell-to-cell registration process may further include performing (606) a tile-level segmentation of the tile-level aligned cross-modality visualization images, in which the tile-level segmentation may be performed to segment each tissue cell in the tile-level aligned cross-modality visualization images. In certain embodiments, the cross-modality cell-to-cell registration process may further include performing (608) an object-level cell registration based on the tile-level segmented tissue cells, in which the object-level cell registration is performed to match individual tissue cells across the cross-modality visualization images. For example, in certain embodiments, the object-level cell registration may include matching, for example, polygons (e.g., 2D polygons or 3D polygons) multi-directionally based on an identification of overlapping tissue cells and/or intersecting tissue cells across the cross-modality visualization images.

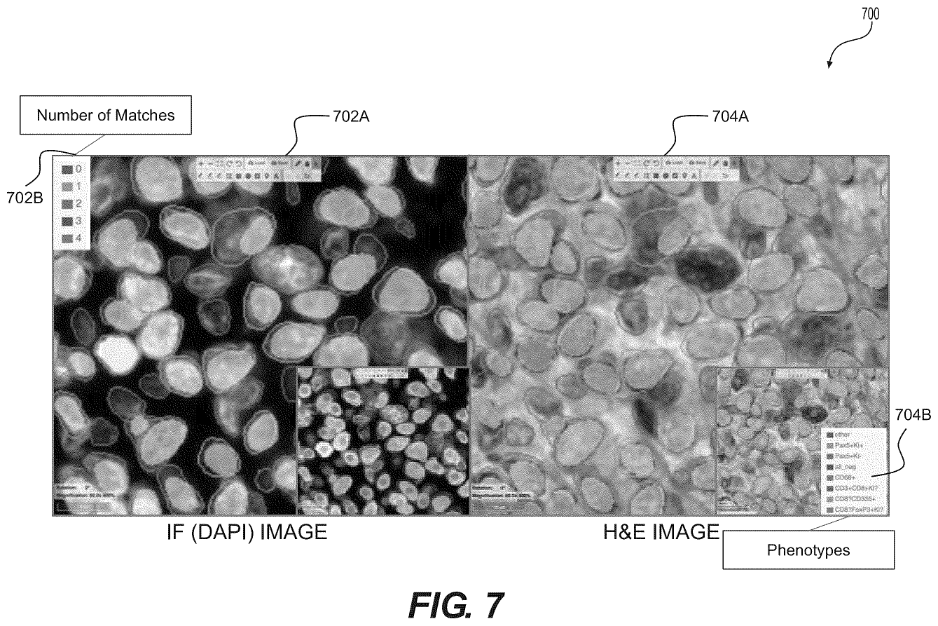

FIG. 7 illustrate implementation examples 700 of the cross-modality cell-to-cell registration process that thus illustrates that the tissue cells may be accurately matched, for example, between mxIF images and H&E images, or across any of various visualization modalities, in accordance with the presently disclosed embodiments. For example, referring to FIG. 7, the implementation example first image 704A illustrates the object-level cell registration of one or more tissue cells between mxIF and H&E visualization modalities as illustrated by the overlapping and/or intersecting polygons and/or outlines corresponding to the mxIF and H&E visualization modalities, respectively. For example, in some embodiments, the implementation example first image 702A illustrates an alignment of segmentation polygons, in which H&E segmentation is shown in yellow (e.g., filled polygons). Similarly, the implementation example first image 704A further illustrates an IF nuclear segmentation of a DAPI (4′, 6-diamidino-2-phenylindole) channel shown as outlines, in which a particular color of the outlines indicates number of matches. Specifically, the implementation example first image 702A illustrates a DAPI channel with phenotype inset and the implementation example second image 704A illustrates an H&E image with the same segmentation overlays.

In certain embodiments, as further depicted. a phenotyping table 702B may also be included and associated with the implementation example first image 702A. In certain embodiments, the phenotyping table 702B may include a list of potential phenotypes that may be color-coded to correspond to the visual overlapping and/or intersecting polygons and/or outlines corresponding to the mxIF and H&E visualization modalities, and may also include a number indicating the number of matches of each different phenotype. Similarly, another phenotyping table 704B may also be included and associated with the implementation example first image 704A. In certain embodiments, the phenotyping table 704B may include a list of potential phenotypes that may be color-coded to correspond to the visual overlapping and/or intersecting polygons and/or outlines corresponding to the mxIF and H&E visualization modalities, and may also include a label (e.g., “other”, “Pax5+Ki+”, “Pax5+Ki−”, “all_neg”, “CD68+”, “CD3+CD3+Ki?”, “CD8?CD335+”, “CD8?FoxP3+Ki?”) indicating each different phenotype. In one embodiment, the “+” or “?” may include an imported symbol representation, which may indicate that additional characters may be included as part of one or more phenotype labels. It should be further appreciated that the smaller images included in the bottom, right corner of the implementation example first image 702A and the implementation example second image 704A, respectively, including an overlay of mapped phenotypes shown as colored dots, for example.

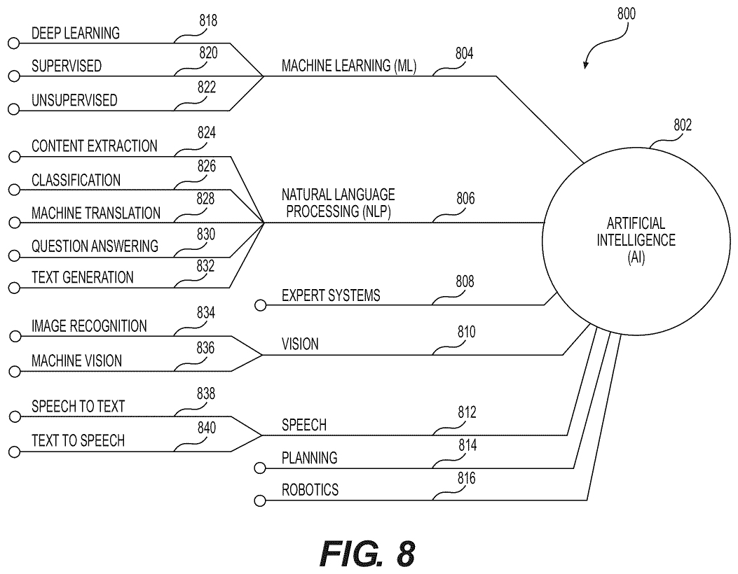

FIG. 8 illustrates a diagram 800 of an example artificial intelligence (AI) architecture 802 (which may be included as part of one or more of the network 100A of interacting computer systems as discussed above with respect to FIG. 1) that may be utilized for performing a cross-modality cell-to-cell registration process to identify, phenotype, and keep track of tissue cells captured utilizing various visualization modalities, according to embodiments of the disclosure. The identification and phenotyping of tissue cells may use one visualization modality and be utilized as verifiable ground truth data for training one or more machine-learning models to classify and phenotype tissue cells captured utilizing another visualization modality, in accordance with the presently disclosed embodiments.

In certain embodiments, the AI architecture 802 may be implemented utilizing, for example, one or more processing devices that may include hardware (e.g., a general purpose processor, a graphic processing unit (GPU), an application-specific integrated circuit (ASIC), a system-on-chip (SoC), a microcontroller, a field-programmable gate array (FPGA), a central processing unit (CPU), an application processor (AP), a visual processing unit (VPU), a neural processing unit (NPU), a neural decision processor (NDP), a deep learning processor (DLP), a tensor processing unit (TPU), and/or other processing device(s) that may be suitable for processing various omics data and making one or more decisions based thereon), software (e.g., instructions running/executing on one or more processing devices), firmware (e.g., microcode), or some combination thereof.

In certain embodiments, as depicted by FIG. 8, the AI architecture 802 may include machine learning (ML) algorithms and functions 804, natural language processing (NLP) algorithms and functions 806, expert systems 808, computer-based vision algorithms and functions 810, speech recognition algorithms and functions 812, planning algorithms and functions 814, and robotics algorithms and functions 816. In certain embodiments, the ML algorithms and functions 804 may include any statistics-based algorithms that may be suitable for finding patterns across large amounts of data (e.g., “Big Data” such as genomics data, proteomics data, metabolomics data, metagenomics data, transcriptomics data, and/or other omics data). For example, in certain embodiments, the ML algorithms and functions 804 may include deep learning algorithms 818, supervised learning algorithms 820, and unsupervised learning algorithms 822.

In certain embodiments. the deep learning algorithms 818 may include any artificial neural networks (ANNs) that may be utilized to learn deep levels of representations and abstractions from large amounts of data. For example, the deep learning algorithms 818 may include ANNs, such as a perceptron, a multilayer perceptron (MLP), an autoencoder (AE), a convolution neural network (CNN), a recurrent neural network (RNN), long short term memory (LSTM), a grated recurrent unit (GRU), a restricted Boltzmann Machine (RBM), a deep belief network (DBN), a bidirectional recurrent deep neural network (BRDNN), a generative adversarial network (GAN), and deep Q-networks, a neural autoregressive distribution estimation (NADE), an adversarial network (AN), attentional models (AM), a spiking neural network (SNN), deep reinforcement learning, and so forth.

In certain embodiments, the supervised learning algorithms 820 may include any algorithms that may be utilized to apply, for example, what has been learned in the past to new data using labeled examples for predicting future events. For example, starting from the analysis of a known training data set, the supervised learning algorithms 820 may produce an inferred function to make predictions about the output values. The supervised learning algorithms 620 may also compare its output with the correct and intended output and find errors in order to modify the supervised learning algorithms 820 accordingly. On the other hand, the unsupervised learning algorithms 822 may include any algorithms that may applied. for example. when the data used to train the unsupervised learning algorithms 822 are neither classified nor labeled. For example, the unsupervised learning algorithms 822 may study and analyze how systems may infer a function to describe a hidden structure from unlabeled data.

In certain embodiments, the NLP algorithms and functions 806 may include any algorithms or functions that may be suitable for automatically manipulating natural language, such as speech and/or text. For example, in some embodiments, the NLP algorithms and functions 806 may include content extraction algorithms or functions 824, classification algorithms or functions 826, machine translation algorithms or functions 828, question answering (QA) algorithms or functions 830, and text generation algorithms or functions 832. In certain embodiments, the content extraction algorithms or functions 824 may include a means for extracting text or images from electronic documents (e.g., webpages, text editor documents, and so forth) to be utilized, for example, in other applications.

In certain embodiments, the classification algorithms or functions 826 may include any algorithms that may utilize a supervised learning model (e.g., logistic regression, naïve Bayes, stochastic gradient descent (SGD), k-nearest neighbors, decision trees, random forests, support vector machine (SVM), and so forth) to learn from the data input to the supervised learning model and to make new observations or classifications based thereon. The machine translation algorithms or functions 828 may include any algorithms or functions that may be suitable for automatically converting source text in one language, for example, into text in another language. The QA algorithms or functions 830 may include any algorithms or functions that may be suitable for automatically answering questions posed by humans in, for example, a natural language, such as that performed by voice-controlled personal assistant devices. The text generation algorithms or functions 832 may include any algorithms or functions that may be suitable for automatically generating natural language texts.

In certain embodiments, the expert systems 808 may include any algorithms or functions that may be suitable for simulating the judgment and behavior of a human or an organization that has expert knowledge and experience in a particular field (e.g., stock trading, medicine, sports statistics, and so forth). The computer-based vision algorithms and functions 810 may include any algorithms or functions that may be suitable for automatically extracting information from images (e.g., photo images, video images). For example, the computer-based vision algorithms and functions 810 may include image recognition algorithms 834 and machine vision algorithms 836. The image recognition algorithms 834 may include any algorithms that may be suitable for automatically identifying and/or classifying objects, places, people, and so forth that may be included in, for example, one or more image frames or other displayed data. The machine vision algorithms 836 may include any algorithms that may be suitable for allowing computers to “see,” or, for example, to rely on image sensors cameras with specialized optics to acquire images for processing, analyzing, and/or measuring various data characteristics for decision making purposes.

In certain embodiments, the speech recognition algorithms and functions 812 may include any algorithms or functions that may be suitable for recognizing and translating spoken language into text, such as through automatic speech recognition (ASR), computer speech recognition, speech-to-text (STT) 838, or text-to-speech (TTS) 840 in order for the computing to communicate via speech with one or more users, for example. In certain embodiments, the planning algorithms and functions 814 may include any algorithms or functions that may be suitable for generating a sequence of actions, in which each action may include its own set of preconditions to be satisfied before performing the action. Examples of Al planning may include classical planning, reduction to other problems, temporal planning, probabilistic planning, preference-based planning, conditional planning, and so forth. Lastly, the robotics algorithms and functions 816 may include any algorithms, functions, or systems that may enable one or more devices to replicate human behavior through, for example, motions, gestures, performance tasks, decision-making, emotions, and so forth.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

Herein, “automatically” and its derivatives means “without human intervention,” unless expressly indicated otherwise or indicated otherwise by context.

The embodiments disclosed herein are only examples, and the scope of this disclosure is not limited to them. Embodiments according to this disclosure are in particular disclosed in the attached claims directed to a method. a storage medium, a system and a computer program product, wherein any feature mentioned in one claim category, e.g., method, may be claimed in another claim category, e.g., system, as well. The dependencies or references back in the attached claims are chosen for formal reasons only. However, any subject matter resulting from a deliberate reference back to any previous claims (in particular multiple dependencies) may be claimed as well, so that any combination of claims and the features thereof are disclosed and may be claimed regardless of the dependencies chosen in the attached claims. The subject-matter which may be claimed comprises not only the combinations of features as set out in the attached claims but also any other combination of features in the claims, wherein each feature mentioned in the claims may be combined with any other feature or combination of other features in the claims. Furthermore, any of the embodiments and features described or depicted herein may be claimed in a separate claim and/or in any combination with any embodiment or feature described or depicted herein or with any of the features of the attached claims.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates certain embodiments as providing particular advantages, certain embodiments may provide none, some, or all of these advantages.

### Embodiments

Among the provided embodiments are:

1. A method for classifying tissue cells into one or more phenotypes, the method comprising, by one or more computing devices: receiving a plurality of images of a set of tissue cells, the plurality of images comprising at least a first image including a first visualization modality and a second image including a second visualization modality; identifying a first tissue cell of the set of tissue cells in the first image and the first tissue cell in the second image: performing a cell-to-cell registration process based on the first tissue cell identified in the first image and the first tissue cell identified in the second image, the cell-to-cell registration process comprising matching of the first tissue cell identified in the first image to the first tissue cell identified in the second image; and classifying the first tissue cell into a phenotype based on the cell-to-cell registration process, the phenotype at least partially indicative of a disease pathology.
2. The method of embodiment 1, further comprising generating a phenotyping table based on the phenotype classification of the first tissue cell.
3. The method of embodiment 2, further comprising mapping the phenotype classification of the first tissue cell identified in the first image to the first tissue cell identified in the second image utilizing the phenotyping table.
4. The method of any one of embodiments 1-3, wherein each of the first visualization modality and the second visualization modality is independently acquired by a whole slide imaging modality, microscopy modality, non-optical imaging modality, or spatial transcriptomics (ST) imaging modality.
5. The method of embodiment 4, wherein the whole slide imaging modality is selected from bright-field or fluorescence imaging.
6. The method of embodiment 4 or 5, wherein the microscopy modality is selected from bright-field microscopy, fluorescence microscopy, confocal microscopy, high-content screening (HCS) microscopy, or synthetic image generation.
7 The method of any one of embodiments 4-6, wherein the non-optical imaging modality is selected from Imaging Mass Cytometry (IMC) or Multiplex Ion Beam Imaging (MIBI).
8. The method of any one of embodiments 4-7, wherein at least one of the first visualization modality and the second visualization modality comprises a dye-based visualization modality.
9. The method of embodiment 8, wherein the dye-based visualization modality is selected from histological staining, fluorescence in situ hybridization (FISH), or immunofluorescence staining.
10. The method of embodiment 9, wherein the histological staining comprises hematoxylin and eosin (H&E) staining or chromogenic staining.
11. The method of any one of embodiments 1-10, wherein at least one of the first visualization modality or the second visualization modality comprises immunostaining.
12. The method of any one of embodiments 1-11, wherein classifying the first tissue cell into the phenotype comprises classifying, based on one or more molecular annotations, a cell state of the first tissue cell as an activated immune cell.
13. The method of any one of embodiments 1-12, wherein the first tissue cell comprises a cancer cell, a plasma cell, a lymphocyte, a macrophage, or a fibroblast.
14. The method of any one of embodiments 1-13, wherein classifying the first tissue cell into the phenotype comprises classifying, based on one or more molecular annotations, the first tissue cell as an immune cell.
15. The method of embodiment 14, wherein the immune cell comprises a macrophage, a regulatory T-cell (Treg), a CD8 cell, a B lymphocyte, or a natural killer (NK) cell.
16. The method of any one of embodiments 1-15, wherein the disease pathology comprises a non-Hodgkin's lymphoma (NHL) disease pathology.
17. The method of embodiment 16, wherein the NHL disease pathology comprises follicular lymphoma (FL).
18. The method of embodiment 16, wherein the NHL disease pathology comprises diffuse large B-cell lymphoma (DLBCL).
19. A method for classifying tissue cells into one or more phenotypes, the method comprising, by one or more computing devices: receiving a plurality of images of a set of tissue cells, the plurality of images comprising at least a first image including a first visualization modality and a second image including a second visualization modality; identifying regions of pixels in the first image and the second image, wherein each of the regions of pixels corresponds to a respective tissue cell of the set of tissue cells: performing a cell-to-cell registration process based on the identified regions of pixels, the cell-to-cell registration process comprising matching a first region of pixels corresponding to a first tissue cell in the first image to a second region of pixels corresponding to the first tissue cell in the second image; and classifying the first tissue cell into a phenotype based on the cell-to-cell registration process, the phenotype at least partially indicative of a disease pathology.
20. The method of embodiment 19, further comprising generating a phenotyping table based on the phenotype classification of the first tissue cell.
21. The method of any one of embodiments 19-20, further comprising: determining a phenotype class label for the first tissue cell based on one or more spatial features associated with the first region of pixels corresponding to the first tissue cell; determining a correspondence between the first region of pixels corresponding to the first tissue cell and the second region of pixels corresponding to the first tissue cell based on the cell-to-cell registration process; and training a model based on 1) the second region of pixels corresponding to the first tissue cell and 2) the determined phenotype class label for the first tissue cell.
22. The method of embodiment 21, further comprising: inputting a third image of the plurality of images to the trained model, the third image including the second visualization modality: and utilizing the trained model to: identify a region of pixels in the third image corresponding to the first tissue cell; and output a predicted phenotype class label for the first tissue cell in the third image, the predicted phenotype class label for the first tissue cell corresponding to the determined phenotype class label for the first tissue cell.
23. The method of embodiment 21 or 22, wherein the model comprises one or more deep neural networks (DNNs).
24. The method of any one of embodiments 21-23, further comprising: determining ground truth data for training the model by mapping the phenotype classification of the first tissue cell to the first tissue cell in the first image and to the first tissue cell in the second image.
25. The method of embodiment 24, wherein at least a subset of the ground truth data comprises molecular annotated ground truth data or wherein at least a subset of the ground truth data comprises human annotated ground truth data.
26. The method of embodiment 24 or 25, further comprising mapping the phenotype classification of the first tissue cell to the first tissue cell in the first image and to the first tissue cell in the second image utilizing a phenotyping table.
27. The method of any one of embodiments 19-26, wherein each of the first visualization modality and the second visualization modality is independently acquired by a whole slide imaging modality, microscopy modality, non-optical imaging modality, or spatial transcriptomics (ST) imaging modality.
28. The method of embodiment 27, wherein the whole slide imaging modality is selected from bright-field or fluorescence imaging.
29. The method of embodiment 27 or 28, wherein the microscopy modality is selected from bright-field microscopy, fluorescence microscopy, confocal microscopy, high-content screening (HCS) microscopy, or synthetic image generation.
30. The method of any one of embodiments 27-29, wherein the non-optical imaging modality is selected from Imaging Mass Cytometry (IMC) or Multiplex Ion Beam Imaging (MIBI).
31. The method of any one of embodiments 27-30, wherein at least one of the first visualization modality and the second visualization modality comprises a dye-based visualization modality.
32. The method of embodiment 31, wherein the dye-based visualization modality is selected from histological staining, fluorescence in situ hybridization (FISH), or immunofluorescence staining.
33. The method of embodiment 32, wherein the histological staining comprises hematoxylin and eosin (H&E) staining or chromogenic staining.
34. The method of any one of embodiments 19-33, wherein at least one of the first visualization modality or the second visualization modality comprises immunostaining.
35. The method of any one of embodiments 19-34, further comprising: prior to identifying the regions of pixels in the first image and the second image, aligning the plurality of images to vertically stack at least the first image and the second image.
36. The method of any one of embodiments 19-35, wherein performing the cell-to-cell registration process comprises performing an alignment of the first image and the second image.
37. The method of embodiment 36, wherein performing the cell-to-cell registration process further comprises performing a tile-level alignment of the first image and the second image, the tile-level alignment comprising a matrix transformation of the alignment of the first image and the second image.
38. The method of embodiment 37, wherein performing the cell-to-cell registration process further comprises performing a tile-level segmentation of the tile-level aligned first image and second image, the tile-level segmentation being performed to segment each tissue cell of the set of tissue cells in the first image and the second image.
39. The method of embodiment 38, wherein performing the cell-to-cell registration process further comprises performing an object-level cell registration based on the tile-level segmented tissue cells, the object-level cell registration being performed to match the first tissue cell in the first image to the first tissue cell in the second image.
40. The method of any one of embodiments 19-39, wherein identifying the regions of pixels in the first image and the second image comprises performing a segmentation of the regions of pixels in the first image and the second image to segment respective tissue cells of the set of tissue cells.
41. The method of any one of embodiments 19-40, further comprising: prior to performing the cell-to-cell registration process, extracting one or more features from the first image and the second image based on the identified regions of pixels, the one or more features utilized to identify the first tissue cell in the first image and the first tissue cell in the second image.
42. The method of any one of embodiments 19-41, wherein classifying the first tissue cell into the phenotype comprises classifying, based on one or more spatial features, the first tissue cell into the phenotype.
43. The method of any one of embodiments 19-42, wherein classifying the first tissue cell into the phenotype comprises classifying, based on one or more molecular annotations, the first tissue cell into the phenotype.
44. The method of any one of embodiments 19-44, wherein the disease pathology comprises a non-Hodgkin's lymphoma (NHL) disease pathology.
45. The method of embodiment 44, wherein the NHL disease pathology comprises follicular lymphoma (FL).
46. The method of embodiment 44, wherein the NHL disease pathology comprises diffuse large B-cell lymphoma (DLBCL).
47. A system including one or more computing devices, comprising: one or more non-transitory computer-readable storage media including instructions: and one or more processors coupled to the one or more storage media, the one or more processors configured to execute the instructions to perform the method of any one of embodiments 1-46.
48. A non-transitory computer-readable medium comprising instructions that, when executed by one or more processors of one or more computing devices. cause the one or more processors to perform the method of any one of embodiments 1-46.

1. A method for classifying tissue cells into one or more phenotypes, the method comprising, by one or more computing devices:
receiving a plurality of images of a set of tissue cells, the plurality of images comprising at least a first image including a first visualization modality and a second image including a second visualization modality;
identifying a first tissue cell of the set of tissue cells in the first image and the first tissue cell in the second image;
performing a cell-to-cell registration process based on the first tissue cell identified in the first image and the first tissue cell identified in the second image, the cell-to-cell registration process comprising matching of the first tissue cell identified in the first image to the first tissue cell identified in the second image; and
classifying the first tissue cell into a phenotype based on the cell-to-cell registration process, the phenotype at least partially indicative of a disease pathology.

2. The method of claim 1, further comprising generating a phenotyping table based on the phenotype classification of the first tissue cell.

3. The method of claim 2, further comprising mapping the phenotype classification of the first tissue cell identified in the first image to the first tissue cell identified in the second image utilizing the phenotyping table.

4. The method of claim 1, wherein each of the first visualization modality and the second visualization modality is independently acquired by a whole slide imaging modality, microscopy modality, non-optical imaging modality, or spatial transcriptomics (ST) imaging modality.

5. The method of claim 4, wherein the whole slide imaging modality is selected from bright-field or fluorescence imaging.

6. The method of claim 4, wherein the microscopy modality is selected from bright-field microscopy, fluorescence microscopy, confocal microscopy, high-content screening (HCS) microscopy, or synthetic image generation.

7. The method of claim 4, wherein the non-optical imaging modality is selected from Imaging Mass Cytometry (IMC) or Multiplex Ion Beam Imaging (MIBI).

8. The method of claim 4, wherein at least one of the first visualization modality and the second visualization modality comprises a dye-based visualization modality.

9. The method of claim 8, wherein the dye-based visualization modality is selected from histological staining, fluorescence in situ hybridization (FISH), or immunofluorescence staining.

10. The method of claim 9, wherein the histological staining comprises hematoxylin and eosin (H&E) staining or chromogenic staining.

11. The method of claim 1, wherein at least one of the first visualization modality or the second visualization modality comprises immunostaining.

12. The method of claim 1, wherein classifying the first tissue cell into the phenotype comprises classifying, based on one or more molecular annotations, a cell state of the first tissue cell as an activated immune cell.

13. The method of claim 1, wherein the first tissue cell comprises a cancer cell, a plasma cell, a lymphocyte, a macrophage, or a fibroblast.

14. The method of claim 1, wherein classifying the first tissue cell into the phenotype comprises classifying, based on one or more molecular annotations, the first tissue cell as an immune cell.

15. The method of claim 14, wherein the immune cell comprises a macrophage, a regulatory T-cell (Treg), a CD8 cell, a B lymphocyte, or a natural killer (NK) cell.

16. The method of claim 1, wherein the disease pathology comprises a non-Hodgkin's lymphoma (NHL) disease pathology.

17. The method of claim 16, wherein the NHL disease pathology comprises follicular lymphoma (FL).

18. The method of claim 16, wherein the NHL disease pathology comprises diffuse large B-cell lymphoma (DLBCL).

19. A system including one or more computing devices, comprising:
one or more non-transitory computer-readable storage media including instructions; and
one or more processors coupled to the one or more storage media, the one or more processors configured to execute the instructions to:
receive a plurality of images of a set of tissue cells, the plurality of images comprising at least a first image including a first visualization modality and a second image including a second visualization modality;
identify a first tissue cell of the set of tissue cells in the first image and the first tissue cell in the second image;
perform a cell-to-cell registration process based on the first tissue cell identified in the first image and the first tissue cell identified in the second image, the cell-to-cell registration process comprising matching of the first tissue cell identified in the first image to the first tissue cell identified in the second image; and
classify the first tissue cell into a phenotype based on the cell-to-cell registration process, the phenotype at least partially indicative of a disease pathology.

20.-36. (canceled)

37. A non-transitory computer-readable medium comprising instructions that, when executed by one or more processors of one or more computing devices, cause the one or more processors to:
receive a plurality of images of a set of tissue cells, the plurality of images comprising at least a first image including a first visualization modality and a second image including a second visualization modality;
identify a first tissue cell of the set of tissue cells in the first image and the first tissue cell in the second image;
perform a cell-to-cell registration process based on the first tissue cell identified in the first image and the first tissue cell identified in the second image, the cell-to-cell registration process comprising matching of the first tissue cell identified in the first image to the first tissue cell identified in the second image; and
classify the first tissue cell into a phenotype based on the cell-to-cell registration process, the phenotype at least partially indicative of a disease pathology.

38.-138. (canceled)
